(19) United States
(12) Patent Application Publication
(10) Pub. No.: US 2009/0192097 A1
(43) Pub. Date: Jul. 30, 2009

(54) Synergistic Herbal Composition from Bacopa Species for Management of Neurodegenerative Disorders and a Process of Preparation Thereof

(76) Inventor: Amit Agarwal, Banaglore (IN)

Correspondence Address: HARNESS, DICKEY & PIERCE, P.L.C., P.O. BOX 828, BLOOMFIELD HILLS, MI, 48303, US

(21) Appl. No.: 12/302,932

(22) PCT Filed: Jun. 5, 2007
(86) PCT No.: PCT/IN2007/000224

§ 371 Date: Dec. 1, 2008

(30) Foreign Application Priority Data
Jun. 7, 2006 (IN) 00977/CHE/2006

(51) Int. Cl. A61K 31/704; A61P 25/28

(52) U.S. Cl. 514/26

(57) ABSTRACT

The present invention relates to a potent synergistic herbal composition [BacoMind®] from the plant species Bacopa monnieri and its beneficial effects in learning, memory, cognition and Attention Deficit Hyperactivity Disorder [ADHD] or Attention Deficit Disorder [ADD]. In addition, the present invention provides the synergistic composition derived from Bacopa monnieri such that the resulting composition consists of Bacoside A3 in the range of 0.1 to 25%, Bacopaside II in the range of 0.1 to 25%, Jujubogenin isomer of bacopasaponin C in the range of 0.1 to 25%, Bacopasaponin C in the range of 0.1 to 25%, Bacopaside I in the range of 0.1 to 25%, Bacosine in the range of 0.1 to 25%, Apigenin in the range of 0.05 to 5%, Luteolin in the range of 0.05 to 5% and Sitosterol-D-glucoside in the range of 0.05 to 5% constituting up to 50% by weight of the total composition.

### FIELD OF THE INVENTION

The present invention relates to a synergistic herbal composition for improving cognition, learning and memory and a process of preparation thereof. More particularly, the present invention relates to a synergistic and enriched herbal composition obtained from the plant Bacopa monnieri Linn. The present invention also relates to a process for the preparation of herbal composition in such a manner that it contains a specific amount of bioactive constituents. The synergistic composition of the present invention is useful in enhancing cognition, improving learning and memory in slow learners and management of neuro-degenerative disorders. It is also useful in management of attention deficit disorders (ADD) and attention deficit hyperactivity disorders (ADHD).

### BACKGROUND OF THE INVENTION

Aging is accompanied by a decline in episodic memory performance seen in middle age (Nilsson L G, 2003 “Memory function in normal aging”, Acta Neurologica Scandinaviaca Supplementum 179:7-13). Memory loss has a high prevalence in the elderly, reputed as occurring in almost half the population aged over 65 years (Reference may be made to Small G W, 2002. “What we need to know about age related memory loss” Brit Med J 324(22): 1502-1505). In USA, prevalence of dementia doubles every 5 years in senior population, thus by the age of 85 about 50% of the individuals develop some degree of cognitive impairment (Reference to the made to Lyons W L, Johnston C B, Covinsky K E, Resnick N M, 2001. Geriatric medicine. In: Current medical diagnosis and treatment. Tierney L M Jr, et. al. (Ed.), Mc Graw Hill, New York, 51). Based on the severity, there are three main forms of memory loss viz., Age Associated Memory Impairment (AAMI), Mild Cognitive Impairment (CMI) and Dementia, in the ascending order. Over the last decade there has been increasing attention in research to try to identify preventive strategies to slow the progression of AAMI and age related cognitive decline, thereby identifying the tools that may delay the onset of dementia.

Dementia is described as a loss of mental function, usually associated with old age, involving problems with memory and reasoning. Dementia interferes with a person's ability to function normally at work as well as in social settings. It is characterized by impairment of short/long-term memory and disintegration of personality due to impaired insight and judgment.

Mild cognitive impairment, or memory problems, is a major risk factor for Alzheimer's disease and other forms of dementia, estimated to affect around 27.7 million people worldwide. According to another study, presented at an international conference, the worldwide direct costs of Alzheimer's disease and dementia care amount to $156 billion. Reference may be made to Alzheimer's Association International Conference on Prevention of Dementia Jun. 18-21, 2005, in Washington, D.C.

The symptoms of dementia are not the result of old age. Severe memory loss is never a normal part of growing older. Dementia symptoms may be static or progressive depending on the underlying disease, and how it is treated. Static dementia usually follows a single major injury like a severe head trauma or heart attack. It does not progress in severity, but remains stable. Progressive dementia, however, does become worse over time. This type of dementia is found in several major brain disorders. Whether it occurs suddenly or gradually, dementia causes many disabling symptoms, including memory disturbance, personality change, impairment of judgment and control of impulses, confusion or disorientation, depression, paranoia or anxiety, diminishing initiative, deterioration of intellectual capacity, obsessive behavior or paranoia, delusions or psychotic episodes.

It is also clear that, as the population of older people grows throughout the world, the number of people with dementia will rise. If current population trends continue, the number of people with dementia could double every 20 years. Currently the exact causes of dementia are not known for certain, and there is no known cure.

### The Biological Basis of Dementia:

Human brain contains hundreds of billions of nerve cells (neurons), any one of which can have thousands or hundreds of thousands of connections with other neurons. Within and between these cells travel dozens of chemical messengers—neurotransmitters, hormones and growth factors, which allow each neuron to exchange information with its neighbors in a vast communications network. Somewhere in this complex system lies the cause of dementia. Dementia is thought to disrupt thinking and memory by affecting the transfer of information between neurons. The present day belief is that there is a reduction of overall cholinergic activity in the brain and most of the modern day therapies aim at enhancing cholinergic transmission.

### Attention Deficit Hyperactivity Disorder [ADHD] or Attention Deficit Disorder [ADD]

ADD/ADHD is one of the most common health issues facing our society today. True, child behavior is a factor, but ADD/ADHD is much more than just a child behavior problem. ADD/ADHD affects up to 15% of our children. Attention Deficit Hyperactivity Disorder (ADHD) is a mental condition that is under-recognized. It often leaves children and teenagers ‘stigmatized’ and undervalued. Although the condition affects mainly boys, girls also can be affected. Parents often are left to cope, whilst their child struggles and is often misunderstood.

ADHD is generally first diagnosed during the primary school years. Symptoms are always present before the age of seven, but sometimes continue into adolescence. Symptoms often become less severe in late teenage and in early adulthood, although it seems that people do not ‘outgrow’ ADHD, but learn to master strategies to compensate for the symptoms. ADHD is under-recognized, with less than half of the affected individuals receiving appropriate diagnoses, and of those who are diagnosed, few receive appropriate treatment. It is believed that ADHD results when a person has lower levels of certain neuro-chemical transmitters in the brain that regulate motor control, attention, organization, planning ahead and decision making.

### ADD/ADHD Present Therapies

ADHD is usually treated with the aid of stimulant drugs like Ritalin, Concerta and with non-stimulant drugs like Straterra as well as amphetamines, such as Dexedrine and Adderall Amphetamines [Adderall and Dexedrine] which are stimulant drugs containing amphetamine and are used to improve attention span and decreases impulsivity, to stimulate the brain; in children, can be used to treat hyperactivity. Methylphenidate hydrochloride [Ex:Ritalin] is another stimulant medication used to treat Attention Deficit Disorder. Concerta is a central nervous system (CNS) stimulant. Strattera—is the first non-stimulant medication approved for the treatment of ADHD in children, adolescents, and adults.

### Limitations of Current Therapies:

For most stimulant medications, the most common side effect is their addition potential. Many youngsters in particular tend to get addicted to amphetamines for their CNS stimulating effects. The other side effects are loss of appetite, sleeping problems, headaches, restlessness or tremor; anxiety or nervousness; dizziness, dryness of the mouth or an unpleasant taste in the mouth; diarrhea or constipation; or impotence or change in sex drive, stomachaches, blood pressure problems in those with a history of hypertension and nervousness or who consume excessive caffeine and have anxiety. The incidence of side effects can vary widely among the different ADHD medications.

### PRIOR ART OF THE INVENTION

Bacopa monnieri is a creeping annual plant found throughout the Indian sub-continent in wet, damp and marshy areas. This medicinal plant is locally known as Brahmi. Bacopa monnieri has been used by Ayurvedic medical practitioners in India for almost 3000 years and classified as a “medhya rasayana”, a drug used to improve memory and intellect (Medhya). The earliest chronicle mention of Bacopa monnieri is in Charaka Samhitha (6^{th} Century A.D) in which it is mentioned in formulations for the management of a range of mental conditions including anxiety, poor cognition and lack of concentration. Brahmi is currently recognized as an effective treatment of mental illness and epilepsy. In the recent years, there has been some attempt to scientifically validate the traditional benefits of Brahmi by various researchers (Ref: Dhawan B N, Singh H K (1996), Eur Neuropsychopharmacol 6: 144)

In view of the importance of this plant, several systematic chemical examinations of the plant have been carried out by various groups of researchers. (Ref: Chakravarty A K, Garai S, Masuda K, Nakane T, Kawahara N (2003) Chem Pharm Bull 51: 215-217, Chatterji N, Rastogi R P, Dhar M L (1965). Ind J Chem 3: 24-29). In spite of the numerous attempts, the chemical composition of the main constituents of Brahmi remains ambiguous. (Please refer our publication Deepak M, Amit A, “The need for establishing identities of ‘bacoside A and B’, the putative major bioactive saponins of Indian medicinal plants Bacopa monnieri” published in ‘Phytomedicine’, 2004, 11(2-3): 264-268). This article raised many doubts about the authenticity of standardization claims published earlier for Bacopa monnieri based products.

There are several products available both in Indian and International market which are made with Bacopa monnieri crude powder and its extract. Most of these products claim specific content of bacosides. In spite of a number of publications, both on the chemistry and pharmacology of Bacopa monnieri, the exact nature of active principles and their mechanism of action is still unknown. The applicant has found, for the first time, after extensive R&D, that bacoside A is a mixture of 4 major saponins viz., (1) Bacoside A3, (2) Bacopaside II, (3) Jujubogenin isomer of bacopasaponin C and (4) Bacopasaponin C. This work was published by the applicant under the title “Quantitative Determination of the Major Saponin Mixture Bacoside A in Bacopa monnieri by HPLC” in ‘Phytochemical Analysis’, 2005, 16: 24-29, Deepak M, Sangli G K, Aran P C & Amit A.). Further, this research paper also illustrates the method of their quantitative estimation.

In the light of the above publication that bacoside A is not a single compound and the identity of bacoside B is still ambiguous, the claims on the content of bacosides in Bacopa based products, cannot be relied upon. Overall, the available literature on Bacopa monnieri indicates that only Bacosides are the active constituents. The applicant has found scientific evidence contrary to this belief.

### Need for the Invention

The applicant carried out a market survey which clearly indicated that virtually all the available products containing the extracts of Bacopa are standardized to the content of Bacoside A/Bacoside B/Bacosides. However, while performing in-house studies to understand the mechanism of action of Bacopa monnieri, the applicant found that Bacopa monnieri was active in two important in-vitro bioassays used to assess the nootropic activity and other neuro-degenerative disorders. These assays were:
1. Affinity towards 5HT₆ receptor
2. Inhibition of butyrylcholinesterase enzyme

Interestingly “Bacoside A” was found to be poorly active while the Bacopa extract showed better activity indicating that there are constituents other than Bacosides which are responsible for the nootropic activity of Bacopa monnieri.

The above findings prompted the applicant to develop a synergistic phytochemical herbal composition derived from Bacopa monnieri, which would be most active in the above in-vitro assays and therefore more useful in the management of neurodegenerative disorders, ADD/ADHD and assist in memory and cognition enhancement. Conventional extracts do not ensure the presence of all the required bioactive compounds.

### Objects of the Present Invention

The principal object of the present invention is to provide a synergistic herbal composition from plant Bacopa monnieri.

Another object of the present invention is to provide a process for preparation of synergistic herbal composition from plant Bacopa monnieri.

Yet another object of the present invention is to provide a synergistic herbal composition from plant Bacopa monnieri for management of neurodegenerative disorders.

Still another object of the present invention is to provide a synergistic and enriched phytochemical composition derived from Bacopa monnieri which is useful for enhancing cognition, improving learning in slow learners and management of neuro-degenerative disorders.

Still another objective of the present invention is to provide a synergistic and enriched phytochemical composition derived from Bacopa monnieri which is also useful in management of attention deficit disorders (ADD) and attention deficit hyperactivity disorders (ADHD).

Still another objective of the present invention is to provide a synergistic and enriched phytochemical composition derived from Bacopa monnieri which is palatable, safe and effective in small doses such that it can be administrated in any dosage form like capsules, tablets, syrups, lozenges etc

Still another objective of the present invention is to provide a synergistic and enriched phytochemical composition derived from Bacopa monnieri which can be mixed with regular food substances like pizza, bread, health drinks, biscuits, chocolates and the like.

Still another objective of the present invention is to provide a process for the preparation of synergistic and enriched phytochemical composition derived from

Bacopa monnieri.

The above stated objectives of the present invention have been achieved, on the basis of the findings that a herbal composition containing specific amounts of certain specific bioactive constituents can be prepared from the plant Bacopa monnieri such that the synergistic composition is useful for enhancing cognition, improving learning in slow learners and management of neurodegenerative disorders as well as in the management of attention deficit disorders (ADD) and attention deficit hyperactivity disorders (ADHD).

### STATEMENT OF THE INVENTION

Accordingly, the present invention provides a synergistic herbal composition obtained from plant Bacopa species for management of neurodegenerative disorders, said composition comprising bacoside A3 at a concentration ranging from 0.1 to 25% w/w, bacopaside II at a concentration ranging from 0.1 to 25% w/w, jujubogenin isomer of bacopasaponin C at a concentration ranging from 0.1 to 25% w/w, bacopasaponin C at a concentration ranging from 0.1 to 25% w/w, bacopaside I at a concentration ranging from 0.1 to 25% w/w, bacosine at a concentration ranging from 0.1 to 25% w/w, apigenin at a concentration ranging from 0.05 to 5% w/w, sitosterol-D-glucoside at a concentration ranging from 0.05 to 5% w/w, and luteolin at a concentration ranging from 0.05 to 5% w/w of the composition optionally along with pharmaceutically acceptable excipients; a process for preparation of synergistic herbal composition from plant Bacopa monnieri for management of neurodegenerative disorders, said process comprising steps of: (i) powdering the plant parts to obtain powder; (ii) extracting the powder using solvent to obtain an extract; (iii) refluxing followed by concentrating the extract to obtain a residue; (iv) centrifuging the residue in a solvent to separate soluble and insoluble materials; and (v) drying the insoluble material to obtain synergistic herbal composition comprising Bacoside A3 at a concentration ranging from 0.1 to 25% w/w, Bacopaside II at a concentration ranging from 0.1 to 25% w/w, Jujubogenin isomer of bacopasaponin C at a concentration ranging from 0.1 to 25% w/w, Bacopasaponin C at a concentration ranging from 0.1 to 25% w/w, Bacopaside I at a concentration ranging from 0.1 to 25% w/w, Bacosine at a concentration ranging from 0.1 to 25% w/w, Apigenin at a concentration ranging from 0.05 to 5% w/w, Sitosterol-D-glucoside at a concentration ranging from 0.05 to 5% w/w, and Luteolin at a concentration ranging from 0.05 to 5% w/w of the composition; and use of a synergistic herbal composition comprising Bacoside A3 at a concentration ranging from 0.1 to 25% w/w, Bacopaside II at a concentration ranging from 0.1 to 25% w/w, Jujubogenin isomer of bacopasaponin C at a concentration ranging from 0.1 to 25% w/w, Bacopasaponin C at a concentration ranging from 0.1 to 25% w/w, Bacopaside I at a concentration ranging from 0.1 to 25% w/w, Bacosine at a concentration ranging from 0.1 to 25% w/w, Apigenin at a concentration ranging from 0.05 to 5% w/w, Sitosterol-D-glucoside at a concentration ranging from 0.05 to 5% w/w, and Luteolin at a concentration ranging from 0.05 to 5% w/w of the composition to manufacture a medicament for management of neurodegenerative disorders in a subject in need thereof.

### BRIEF DESCRIPTION OF THE ACCOMPANYING DRAWINGS

FIG. 1 TLC showing the degradation of Bacoside A

FIG. 2 HPLC chromatogram of Bacomind showing the presence of Luteolin, Apigenin, Bacopaside I, Bacoside A3, Bacopaside II, Jujubogenin isomer of bacopasaponin C and Bacopasaponin C.

FIG. 2a HPLC chromatogram of Bacomind showing presence of Bacosine

FIG. 2b HPLC Chromatogram showing presence of β-sitosterol-D-glucoside

FIG. 3 Effect of Bacomind on Lipoxygenase activity

FIG. 4 Effect of Bacomind on DPPH radical scavenging activity

FIG. 5 Effect of Bacomind on ABTS radical scavenging activity

FIG. 6 Effect of Bacomind on Butrylcholinesterase activity

### DETAILED DESCRIPTION OF THE PRESENT INVENTION

The present invention relates to a synergistic herbal composition obtained from plant Bacopa species for management of neurodegenerative disorders, said composition comprising bacoside A3 at a concentration ranging from 0.1 to 25% w/w, bacopaside II at a concentration ranging from 0.1 to 25% w/w, jujubogenin isomer of bacopasaponin C at a concentration ranging from 0.1 to 25% w/w, bacopasaponin C at a concentration ranging from 0.1 to 25% w/w, bacopaside I at a concentration ranging from 0.1 to 25% w/w, bacosine at a concentration ranging from 0.1 to 25% w/w, apigenin at a concentration ranging from 0.05 to 5% w/w, sitosterol-D-glucoside at a concentration ranging from 0.05 to 5% w/w, and luteolin at a concentration ranging from 0.05 to 5% w/w of the composition optionally along with pharmaceutically acceptable excipients. In another embodiment of the present invention, wherein the concentration of bacoside A3 is preferably about 5.7% w/w, bacopaside II is preferably about 5.6% w/w, jujubogenin isomer of bacopasaponin C is preferably about 8.2% w/w, bacopasaponin C is preferably about 5.4% w/w, bacopaside I is preferably about 7.1% w/w, bacosine is preferably about 1.9%, apigenin is preferably about 0.3%, sitosterol-D-glucoside is preferably about 0.9% and luteolin is preferably about 0.5%.

Yet another embodiment of the present invention, wherein the Bacopa species is Bacopa monnieri.

Still another embodiment of the present invention, wherein said neurodegenerative disorders comprise attention deficit disorders, attention deficit hyperactivity disorders, dementia, amnesia, alzhemier's disease, cognition and slow learning.

Still another embodiment of the present invention, wherein said pharmaceutically acceptable excipients are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

Still another embodiment of the present invention, wherein the composition is formulated into dosage forms selected from a group comprising tablet, troches, lozenges, aqueous or oily suspensions, dispersible powders or granules, emulsion in hard or soft gel capsules, syrups, elixirs, phyotceuticals, neutraceuticals and food stuffs.

Still another embodiment of the present invention, wherein said composition non-toxic and free of side effects.

The present invention is in relation to a process for preparation of synergistic herbal composition from plant Bacopa monnieri for management of neurodegenerative disorders, said process comprising steps of:

(i) powdering the plant parts to obtain powder;
(ii) extracting the powder using solvent to obtain an extract;
(iii) refluxing followed by concentrating the extract to obtain a residue;
(iv) centrifuging the residue in a solvent to separate soluble and insoluble materials; and
(v) drying the insoluble material to obtain synergistic herbal composition comprising Bacoside A3 at a concentration ranging from 0.1 to 25% w/w, Bacopaside II at a concentration ranging from 0.1 to 25% w/w, Jujubogenin isomer of bacopasaponin C at a concentration ranging from 0.1 to 25% w/w, Bacopasaponin C at a concentration ranging from 0.1 to 25% w/w, Bacopaside I at a concentration ranging from 0.1 to 25% w/w, Bacosine at a concentration ranging from 0.1 to 25% w/w, Apigenin at a concentration ranging from 0.05 to 5% w/w, Sitosterol-D-glucoside at a concentration ranging from 0.05 to 5% w/w, and Luteolin at a concentration ranging from 0.05 to 5% w/w of the composition.

In another embodiment of the present invention wherein said plant parts are selected from a group comprising root, shoot, seeds and leaves or the whole plant.

Yet another embodiment of the present invention, wherein the plant parts are powdered

to obtain particle size ranging from 30 # to 50 # and preferably of 40 #.

Still another embodiment of the present invention, wherein said powder is extracted using alcoholic solvent selected from a group comprising ethanol, methanol, propanol and isopropanol.

Still another embodiment of the present invention, wherein said powder is extracted preferably using methanol.

Still another embodiment of the present invention, wherein said extract is refluxed at temperature ranging from 60 to 80° C. and for time period ranging from 1 to 6 hours.

Still another embodiment of the present invention, wherein said extract is refluxed at temperature preferably about 70° C. and preferably for time period of about 4 hours.

Still another embodiment of the present invention, wherein said extract is concentrated under vacuum to obtain concentrated residue.

Still another embodiment of the present invention, wherein the insoluble material is dried at a temperature less than 75° C.

The present invention is in relation to use of a synergistic herbal composition comprising Bacoside A3 at a concentration ranging from 0.1 to 25% w/w, Bacopaside II at a concentration ranging from 0.1 to 25% w/w, Jujubogenin isomer of bacopasaponin C at a concentration ranging from 0.1 to 25% w/w, Bacopasaponin C at a concentration ranging from 0.1 to 25% w/w, Bacopaside I at a concentration ranging from 0.1 to 25% w/w, Bacosine at a concentration ranging from 0.1 to 25% w/w, Apigenin at a concentration ranging from 0.05 to 5% w/w, Sitosterol-D-glucoside at a concentration ranging from 0.05 to 5% w/w, and Luteolin at a concentration ranging from 0.05 to 5% w/w of the composition to manufacture a medicament for management of neurodegenerative disorders in a subject in need thereof.

In another embodiment of the present invention, wherein the subject is animal including human beings.

Yet another embodiment of the present invention, wherein the composition is administered at a dose ranging from 100 mg to 3000 mg per day or in divided doses or in single dose by the subjects in need thereof.

Still another embodiment of the present invention, wherein the composition is free of adverse effects.

The present invention provides a synergistic and enriched herbal composition useful for enhancing cognition, improving learning in slow learners and management of neuro-degenerative disorders derived from the plant Bacopa monnieri which comprises

(i) Bacoside A3 in an amount in the range of 0.1 to 25% by weight of the composition
(ii) Bacopaside II in an amount in the range of 0.1 to 25% by weight of the composition
(iii) Jujubogenin isomer of bacopasaponin C in an amount in the range of 0.1 to 25% by weight of the composition,
(iv) Bacopasaponin C in an amount in the range of 0.1 to 25% by weight of the composition,
(v) Bacopaside I in an amount in the range of 0.1 to 25% by weight of the composition
(vi) Bacosine in an amount in the range of 0.1 to 25% by weight of the composition
(vii) Apigenin in an amount in the range of 0.05 to 5% by weight of the composition
(viii) Sitosterol-D-glucoside in an amount in the range of 0.05 to 5% w/w by weight of the composition and
(ix) Luteolin in an amount in the range of 0.05 to 5% w/w by weight of the composition and the phytocomponents from point [i] to [ix] put together comprises up to 50% w/w of the composition and the remaining being unknown constituents.

To the best of my knowledge, the synergistic composition described above does not exist any where.

According to another embodiment of the present invention, there is provided a process for the preparation of the synergistic herbal composition useful for enhancing cognition, improving learning in slow learners and management of neuro-degenerative disorders derived from the plant Bacopa monnieri which comprises

(i) cleaning the aerial parts of Bacopa monnieri and powdering it to a mesh size 30-40,
(ii) extracting the resulting coarse powder with alcohol (methanol being preferable),
(iii) refluxing the extract in a water bath for a time period ranging between 1 to 6 hours and filtering,
(iv) adding alcohol (methanol being preferable) to the residue and refluxing again for 2 to 4 hours,
(v) repeating the steps (ii) & (iii)
(vi) mixing the filtrates obtained from the repeated extractions and distilling off the alcohol under vacuum to get a concentrated extract
(vii) adding water to the resulting mixture and stirring for a period in the range of 1 to 2 hours to remove impurities,
(viii) centrifuging or filtering to separate the soluble material from the insoluble material,
(ix) drying the resulting insoluble material in a vacuum tray drier at < 75° C. to get a light green powder and
(x) milling and sieving the resulting powder.

In a preferred embodiment of the invention, the solvent used in step (i) may be selected from ethanol, methanol, propanol and iso-propanol while methanol being the preferred solvent, the period of refluxing in step (ii) may be two hrs, in step (v) the alcohol used is the same as that used in step (ii) the steps (ii) & (iii) may preferably be repeated for at least three times.

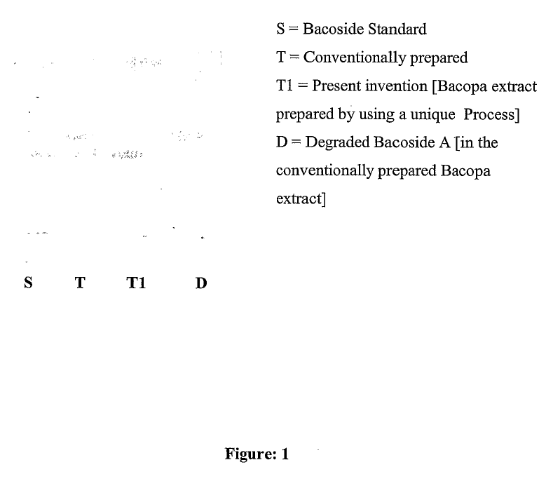

The synergistic herbal composition of the present invention retains the original phytochemistry of the plant Bacopa monnieri. It has been confirmed by studies that the composition prepared, from the said plant in the manner, as described in the published conventional methods and published patent specifications, that the ‘bacosides’ present in the plant get degraded to a considerable extent as shown in FIG. 1 of the drawing accompanying this specification. Reference may be made to U.S. Pat. No. 6,833,143, Indian Patent No. 185078, PCT application No. PCT/IN2003/000002 & PCT application No. PCT/IB2002/005452. The main reason for this degradation is the use of butanol in the extraction process. Butanol being a high boiling point solvent (boiling point 118° C.) requires long hours and higher temperatures during distillation for removing the solvent from the extract.

To the best of our knowledge, there is no Bacopa monnieri based composition hitherto known containing all the above mentioned active components and in the said specific percentages defined above.

Similarly, to the best of our knowledge there is no conventional Bacopa monnieri based composition which claims to contain all nine bio-active compounds in the specified range i.e., Bacoside A3 in an amount in the range of 0.1 to 25% by weight of the composition, Bacopaside II in an amount in the range of 0.1 to 25% by weight of the composition, Jujubogenin isomer of bacopasaponin C in an amount in the range of 0.1 to 25% by weight of the composition, Bacopasaponin C in an amount in the range of 0.1 to 25% by weight of the composition, Bacopaside I in an amount in the range of 0.1 to 25% by weight of the composition, Bacosine in an amount in the range of 0.1 to 25% by weight of the composition, Apigenin in an amount in the range of 0.05 to 5% by weight of the composition, Sitosterol-D-glucoside in an amount in the range of 0.05 to 5% w/w by weight of the composition and Luteolin in an amount in the range of 0.05 to 5% w/w by weight of the composition and these all nine phytocomponents, put together comprises up to 50% w/w of the composition and the remaining being unknown.

Since, the process for the preparation of the composition of the present invention involves only alcohol which is very easy to remove during industrial distillation; the bioactive compounds including Bacosides do not degrade in our process. Moreover, the enrichment according to the process of the present invention involves only removal of impurities with water and discarding the water layer. No further distillations are required and hence the product does not undergo any degradation.

Accordingly, the present invention prevents the degradation of Bacoside A as depicted in the chromatogram as shown in FIG. 1. In an embodiment of the present invention, the synergistic herbal extract composition may be mixed with GRAS (Generally Regarded As Safe) grade of pharmaceutical excipients to prepare various dosage forms like capsules, tablets, syrups, lozenges etc. The excipients which can be used may be selected from starch, di-calcium phosphate, polysorbate, fumed silica, etc. In an another embodiment of the present invention, the synergistic herbal composition may be used as a dietary supplement/food additive and thus may be mixed with different foods like bread, pizza, health drinks, biscuits, chocolates, Pasta etc. In yet another embodiment of the present invention the synergistic herbal extract composition may be administered to humans between 0.1 g to 3.0 g per day in divided doses or as a single dose.

The technology of the instant Application is further elaborated with the help of following examples. However, the examples should not be construed to limit the scope of the invention.

### Example: 1

100 grams of powdered leaves of Bacopa monnieri were taken and extracted with 300 ml of ethanol. The extract is refluxed in a water bath for 2 hours at 78° C. temperature and filtered through whatman-41 filter paper. To the residue obtained, another 200 ml of ethanol was added and refluxed for another 2 hours. The above procedure was repeated three times. The filtrates obtained from 3 extractions were distilled under vacuum in a rotary evaporator. 150 ml of water was added to the residue (remained after distillation) and stirred for 1 hour to remove impurities. The contents were subjected to centrifugation to separate the soluble and insoluble materials. The insoluble material was dried in a vacuum tray drier at < 75° C. to obtain 5 g of light green powder which contained the following bioactive phytocompounds in the amounts shown against each (all % by weight of the composition)

| | | |
|---|---|---|
| 1. | Bacoside A3 | 9.2% |
| 2. | Bacopaside II | 8.6% |
| 3. | Jujubogenin isomer of bacopasaponin C | 7.8% |
| 4. | Bacopasaponin C | 8.7% |
| 5. | Bacopaside I | 7.5% |
| 6. | Bacosine | 0.8% |
| 7. | Apigenin | 2.5% |
| 8. | Luteolin | 1.2% |
| 9. | Sitosterol-D-glucoside | 2.0% |

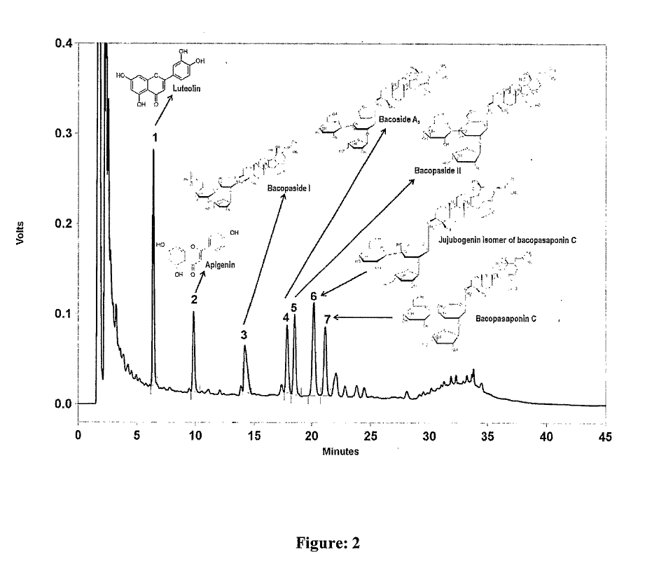

and all 9 compounds put together totaling to 48.3% w/w and the remaining being the unknown compounds. All the components of the composition are characterized using HPLC and HPTLC of which the FIG. 2 provides the HPLC chromatogram for Bacomind showing the presence of Luteolin, Apigenin, Bacopaside I, Bacoside A3, Bacopaside II, Jujubogenin isomer of bacopasaponin C and Bacopasaponin C. In addition, FIG. 2a provides HPLC chromatogram of Bacomind showing presence of Bacosine and FIG. 2b provides HPTLC Chromatogram showing presence of (3-sitosterol-D-glucoside. Here, some of the components were characterized using HPLC and only one compound by name (3-sitosterol-D-glucoside is characterized by HPTLC.

### Example: 2

100 grams of powdered aerial parts of Bacopa monnieri were taken and extracted with 600 ml of methanol. The extract was refluxed on a water bath for 4 hours at 65° C. temperature and filtered through 3 micron filter fabric under vacuum. To residue, another 400 ml of methanol was added and refluxed for another 4 hours. The above procedure was repeated twice. The filtrates obtained from both extractions were distilled under vacuum in a rotary evaporator. 200 ml of water was added to the residue (remained after distillation) and stirred for 1 hour to remove impurities. The contents were subjected to centrifugation to separate the soluble from insoluble materials. The insoluble material was vacuum tray dried and powdered to get 4.7 g of light green powder which was found to contain following phytocompounds in the amount shown against each (all % by weight of the composition)

| | | |
|---|---|---|
| 1. | Bacoside A3 | 5.8% |
| 2. | Bacopaside II | 6.4% |
| 3. | Jujubogenin isomer of bacopasaponin C | 7.1% |
| 4. | Bacopasaponin C | 4.6% |
| 5. | Bacopaside I | 7.5% |
| 6. | Bacosine | 1.7% |
| 7. | Apigenin | 0.2% |
| 8. | Luteolin | 0.8% |
| 9. | Sitosterol-D-glucoside | 0.55% |

and all 9 compounds put together totaling to 34.65% w/w and the remaining being the unknown compounds. The compounds characterized are provided in FIGS. 2, 2a and 2b.

### Example 3

10 Kgs of Bacopa monnieri powdered aerial parts were taken and extracted with 40 liters of methanol. The extract was refluxed in a suitable jacketed vessel for 3 hours at 65° C. temperature and filtered through an online filter. To the residue, another 30 liters of methanol was added and refluxed for another 3 hours. The above procedure was repeated two more times. The filtrates obtained from 2 extractions were distilled under vacuum. 20 liters of water was added to the residue (remained after distillation) and stirred for 1 hour. The contents were subjected to filtration in a filterpress and soluble and insoluble materials were separated. The insoluble material is dried in vacuum tray drier at <70° C. to get dried lumps (530 g). These lumps were milled and sieved. The resulting composition comprises (% by weight)

| | | |
|---|---|---|
| 1. | Bacoside A3 | 5.7% |
| 2. | Bacopaside II | 5.6% |
| 3. | Jujubogenin isomer of bacopasaponin C | 8.2% |
| 4. | Bacopasaponin C | 5.4% |
| 5. | Bacopaside I | 7.1% |
| 6. | Bacosine | 1.9% |
| 7. | Apigenin | 0.3% |
| 8. | Luteolin | 0.5% |
| 9. | Sitosterol-D-glucoside | 0.9% |

and all 9 compounds put together totaling to 35.6% w/w and the remaining being the unknown compounds. The compounds characterized are provided in FIGS. 2, 2a and 2b.

For the purpose of research and further development, Example 3 was selected and trademarked as BacoMind®.

BacoMind® was further tested in butyrylcholinesterase inhibition assay (BCIA) and the results are shown in table 2.

### Example: 4

In order to verify the synergy amongst the phytocompounds present in the present invention, the composition was tested in a bioassay called “5-HT₆ receptor binding assay”. The assay is believed to be relevant to nootropic activity. This assay also indicates the utility of the present invention in neuro-degenerative disorders, ADD/ADHD and improvement in learning and memory with reference to slow learning children in particular.

### 5-HT₆ Receptor Binding Assay:

Antagonists of serotonin receptors (5-HT₆) have been reported to enhance cognition in animal models of learning. The 5-HT₆ receptor was first isolated from rat striatal mRNA in 1993. It is localized almost exclusively in the central nervous system, including areas important for learning and memory, such as the cerebral cortex and hippocampus. The suggestion that 5-HT₆ receptor antagonists may have therapeutic potential as novel treatments for cognitive deficits is supported by reports that they facilitate cholinergic and glutamatergic neurotransmission.

Several studies have reported that specific 5-HT₆ receptor antagonists improve learning and memory in animal models. Analogs of the selective 5-HT₆ receptor antagonist Ro 04-6790 attenuated scopolamine-induced deficits in a passive avoidance task. (Ref: Lindner M D, Hodges D B Jr, Hogan J B, Orie A F, Corsa J A, Barten D M, Poison C, Robertson B J, Guss V L, Gillman K W, Starrett J E Jr, Gribkoff V K, J Pharmacol Exp Ther. 2003; 307(2):682-91).

### 5-HTf₆ Receptor Binding Assay of the Present Composition

The receptor binding assay was done in a filtration format. A total reaction volume of 250 μl contained—5-HT₆ receptor membranes (5 μg/ml obtained from receptors expressed in recombinant HEK 293 cells), varying concentration of the invention or Methiothepin, ³H-LSD 1.7 nM in a suitable binding buffer. The reaction was incubated for 60 minutes at 37° C. Following incubation, vacuum filtration was done to terminate the reaction using GF/C filter and washed several times using 250 μl of ice cold buffer. The radioactivity was counted in a scintillation counter—Chameleon Plate reader (Hidex, Finland). The percentage inhibition of radioligand binding by the sample/positive control was calculated (table 01) Methiothepin at 10 μm/ml was used for nonspecific binding. Determinations were done in duplicate.

TABLE 1
showing 5-HT₆ receptor binding activity
of present composition vs. Bacosides A

| Tested material | Concentration in μg/ml | Average % inhibition |
|---|---|---|
| Present invention | 500 | 92.16 |
| | 100 | 90.26 |
| | 50 | 88.15 |
| | 10 | 40.33 |
| | 5.0 | 21.6 |
| | 0.5 | 9.29 |
| Bacosides A coded as | 50 | 55.32 |
| NR/BM/02 | 100 | 62.64 |
| | 500 | 80.02 |
| Methiothepin | 100 nM | 84.53 |
| (positive Control) | | |

Present invention showed significant affinity to 5-HT₆ receptors. This may explain the possible mechanism by which the composition might be working to augment learning and memory observed in-vivo and in clinical trials. As can be seen from the Table 1, Bacoside A showed poor affinity to 5-HT₆ receptors when compared to the present invention.

### Example: 5

### Butyrylcholinesterase and Memory

Progressive dysfunction of cholinergic neurotransmission in the brain contributes to loss of memory. Loss of cholinergic cells, particularly in the basal forebrain, is accompanied by loss of the neurotransmitter acetylcholine. Butyryl cholinesterase (BuChE) is an enzyme that is related to Acetylcholinesterase (AChE). It is expressed in glia, endothelial cells, and in neurons in selected areas of the central and peripheral nervous systems. Although the function of BuChE remains to be clearly defined, this enzyme is capable of catalyzing the hydrolysis of acetylcholine, and inhibition of BuChE leads to increased levels of this neurotransmitter in the brain.

In dementia, while the level of AChE is decreased, the level of BuChE is increased. In particular, high levels of BuChE are found associated with neuritic plaques and neurofibrillary tangles. It has been speculated that under pathologic conditions, cholinesterases, and in particular BuChE, may play a role in maturation of neuritic plaques. (Ref: Darvesh S, Walsh R, Kumar R, Caines A, Roberts S, Mager D, Rockwood K, Martin E (2003). 17(2): 117-126).

### Butyrylcholinesterase Assay of BacoMind®

Butyrylcholinesterase inhibition assay was carried out using the Ellman's reagent (Ref: Vogel G, Vogel W (2002) Drug discovery and evaluation of pharmacological assays, Springer, New York. 601). In brief, 110 μl of phosphate buffer/BacoMind® solution of various concentrations, 115 μl of DTNB and 25 μl of enzyme are mixed and pre-incubated at room temperature (25° C.) for 5 minutes. Following pre-incubation, 10 μl (6 mM) of butyrylthiocholine iodide substrate is added and mixed. Incubated at room temperature (25° C.) for 5 minutes. The absorbance was read at 405 nm. Table 2: shows the results of Butyryl cholinesterase inhibitory activity of the composition of BacoMind® and bacosides.

TABLE 2
Butyrylcholinesterase inhibition assay
of BacoMind ® vs. Bacoside A

| | | Concentration | | |
|---|---|---|---|---|
| Sl. | | tested | % | IC₅₀ |
| No. | Tested material | (μg/ml) | Inhibition | (μg/ml) |
| 1. | BacoMind ® Batch | 750 | 15.10 | 1698.48 |
| | no.: BM/04010 | 1500 | 27.27 | (1534.47- |
| | | 3000 | 88.98 | 1887.60) |
| 2 | Bacoside A | 1000 | 15.45 | No activity |
| | | 2000 | 22.96 | detected |
| | | 3000 | 17.80 | upto 3 |
| | | | | mg/ml |

The composition of the present invention has shown inhibition and demonstrates that it inhibits butyrylcholinesterase inhibition assay (BCIA). This clearly shows the synergistic effects of the present invention in butyrylcholinesterase inhibition assay (BCIA).

### Example: 6

### Pharmacological Activities to Evaluate Nootropic Activity OF BacoMind® Object Recognition Test in Rats

Objective: To study the nootropic activity of BacoMind® using object recognition test in albino Wistar rats.
Test Method: Forty two albino Wistar rats (150-175 g) of either sex obtained from National Toxicology Centre, Pune were used for the study. The animals were acclimatized for a week and maintained under standard laboratory conditions, given free access to feed (Source: Laboratory prepared) and water, ad libitum.

### Experimental Design:

Six albino Wistar rats of either sex were randomly allotted to each group. Group I served as the vehicle control. Group II, III and IV received BacoMind® orally at the doses of 27, 40 and 54 mg/kg for 7 days. Piracetam was administered to group V (100 mg/kg, i.p, single dose, on 7^{th} day 30 min before first trial) and served as the reference standard control. Group VI was administered with scopolamine (0.3 mg/kg, i.p, single dose, on 7th day 30 min before first trial) and served as the amnesia control. Group VII was administered both with BacoMind™ (40 mg/kg, p.o, for 7 days) and scopolamine (0.3 mg/kg, i.p, single dose, on 7th day 30 min after BacoMind® treatment). The day before testing, the animals were allowed to explore the box for two minutes. On the day of test (30 min after last dose) a session of two trials was given. An intertrial interval of 60 min was maintained. In the first trial (T1), two identical objects were presented in the opposite corners of the box and the amount of time taken by each animal to complete 20 sec of object exploration was recorded. Exploration was considered, directing nose at a distance <2 cm to the object or touching it with nose. During the second trial (T2), one of the objects presented in T1 was replaced by a new object and the animal was left individually in the box for 5 min. The time spent for exploration of the familiar (F) and new (N) objects were recorded and Discrimination Index (D) was calculated.

### Conclusion

Based on the findings of the present study, it can be concluded that the enriched phytochemical composition BacoMind® when given at the doses of 27, 40 and 54 mg/kg b.w. in albino Wistar rats showed significant nootropic activity at all the above doses. BacoMind® when given at the dose of 40 mg/kg orally for 7 days in scopolamine induced amnesic rats showed significant increase in discrimination index when compared to scopolamine control and thereby exhibited the nootropic effect.

### REFERENCES

1. Bartolini L, Casamenti F, Pepeu G. Aniracetam restores object recognition impaired by age, scopolamine and nucleus basalus lesion. Pharmacol. Biochem. Behav., 1996; 53: 277-283.
2. Ennaceur A, Delacour J. A new one trial test for neurobiological studies of memory in rats. Behav. Brain. Res., 1988; 31: 47-59.

### Example: 7

### Elevated Plus Maze (EPM) Test in Mice

Objective: To study the nootropic activity of BacoMind® using elevated plus maze test in albino Swiss mice.

### Test Method:

Forty two albino Swiss mice (20-22 g) of either sex obtained from National Toxicology Centre, Pune were used for the study. The animals were acclimatized for a week and maintained under standard laboratory conditions, given free access to feed (Source: Laboratory prepared) and water, ad libitum.

### Experimental Design:

Six albino Swiss mice of either sex were randomly allotted to each group. Group I served as the vehicle control. Group II, III and IV received BacoMind® orally at the doses of 40, 60 and 80 mg/kg for 7 days. Piracetam was administered to group V (100 mg/kg, i.p, single dose on 7^{th} day, 30 min before recording transfer latency) and served as the reference standard control. Group VI was administered with scopolamine (0.3 mg/kg, i.p, single dose on 7th day, 30 min before recording transfer latency) and served as the amnesia control group. Group VII was administered both with BacoMind™ (60 mg/kg, p.o, for 7 days) and scopolamine (0.3 mg/kg, i.p, single dose on 7th day, 30 min after BacoMind® treatment). The mice were placed individually at the end of open arm of the EPM facing away from the center. The time taken by the mouse to move into the enclosed arm was noted as transfer latency (TL). The mice were treated with the test substance/drugs as shown in the study design. On day 6, before administration of BacoMind® the animals were placed on the EPM and TL was measured. After determination of the TL, mice were allowed to explore the maze for 2 min and then transferred to their home cages. The TL was again measured after 24 h i.e on 7th day. The TL was expressed as inflexion ratio.

### Results:

A significant decrease in the inflexion ratio was noticed in the scopolamine treated group as compared to the vehicle control. BacoMind® administered at the dose of 60 mg/kg in scopolamine treated mice showed significant increase in the inflexion ratio as compared to the scopolamine control (Table 1).

### Conclusion:

Based on the above findings, it can be stated that enriched phytochemical composition of BacoMind® when given at the dose of 60 mg/kg b.w. orally for 7 days in copolamine induced amnesic mice showed memory enhancing effect in elevated plus maze model. Thus indicates the usefulness of BacoMind® enhancing cognition and learning and memory in individuals.

### REFERENCES

Lister R G. The use of plus maze to measure anxiety in mouse. Psychopharmacol., 1987; 92: 180-185.

### Example: 8

### Passive Avoidance Test in Mice

Objective: To study the nootropic activity of BacoMind® using passive shock avoidance test in albino Swiss mice.

### Test System:

Forty two albino Swiss mice (20-22 g) of either sex obtained from National Toxicology Centre, Pune were used for the study. The animals were acclimatized for a week and maintained under standard laboratory conditions, given free access to feed (Source: Laboratory prepared) and water, ad libitum.

### Experimental Design:

Six albino Swiss mice of either sex were randomly allotted to each group. Group I served as the vehicle control. Group II, III and IV received BacoMind™ orally at the doses of 40, 60 and 80 mg/kg for 7 days. Piracetam was administered to group V (100 mg/kg, i.p, single dose, on 7^{th} day 20 min before first trial) and served as the reference standard control. Group VI was administered with scopolamine (0.3 mg/kg, i.p, single dose, on 7th day 20 min before first trial) and served as the amnesia control. Group VII was administered both with BacoMind™ (60 mg/kg, p.o, for 7 days) and scopolamine (0.3 mg/kg, i.p, single dose, on 7th day 30 min after BacoMind™ treatment). Mice were placed individually on the electric grid and allowed to explore for one minute. A stimulus of 20 V with AC current of 5 mA was given and latency to reach SFZ was recorded for three consecutive times and considered as basal reading. After 1 h of the first trial, each animal was placed on the electric grid again and the latency to reach SFZ and the mistakes (descents) the animal made in 15 min were recorded and considered as parameters for acquisition and retention respectively.

### Results:

The reference standard, piracetam significantly decreased the latency to reach SFZ and the number of mistakes in 15 min as compared to the vehicle control. A significant increase in the latency to reach SFZ but not in the mistakes in 15 min was noticed in the scopolamine treated group as compared to the vehicle control group. BacoMind® showed significant decrease in the latency to reach SFZ and the number of mistakes in 15 min at all the tested doses when compared to the vehicle control. BacoMind® administered at the dose of 60 mg/kg in scopolamine treated mice showed significant decrease in latency to reach SFZ and the number of mistakes in 15 min as compared to the scopolamine control.

### Conclusion

The findings of the present study revealed that the enriched phytochemical composition BacoMind® when given at the doses of 40, 60 and 80 mg/kg b.w orally in mice, significantly reduced the latency to reach SFZ and the number of mistakes in 15 minutes and thereby showed the memory enhancing effect. BacoMind™ given at the dose of 60 mg/kg b.w in scopolamine induced amnesic mice exhibited nootropic activity by significantly decreasing the latency to reach SFZ and the number of mistakes in 15 minutes when compared to scopolamine control. This indicates the usefulness of the composition of BacoMind® in enhancing cognition, learning and memory in individuals.

### Example: 9

Further BacoMind® was evaluated in the following bioassays

1. Lipoxygenase inhibition assay
2. DPPH free radical scavenging assay
3. ABTS radical scavenging assay
4. Butyrylcholinesterase inhibition assay

### 1. Lipoxygenase Inhibition Assay

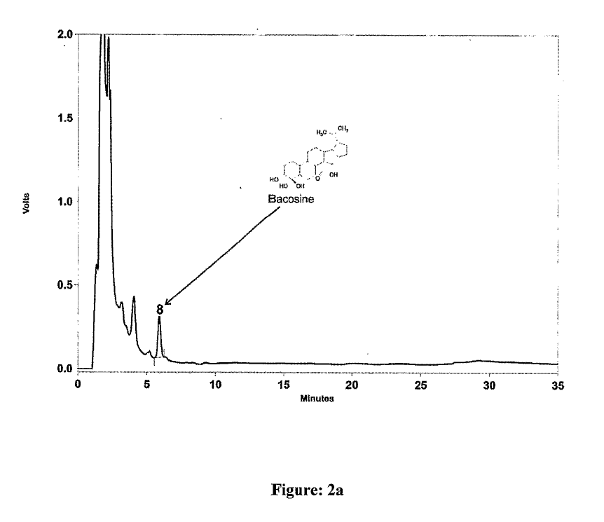

Lipoxygenases (LO) are members of a class of non-heme iron-containing dioxygenases that catalyze the addition of molecular oxygen to fatty acids containing a cis-1,4-pentadiene system to give an unsaturated fatty acid hydroperoxide. In mammals, lipoxygenases carry out the first step in the arachidonic acid cascade [1, 2]. 5- and 15-LOs lead to the biologically active lipoxins, whereas 5-LO leads to 5,6-epoxy-leukotrienes which are involved in a variety of inflammatory responses, including neutrophil chemotaxis, vascular permeability, and smooth muscle contraction [3]. There is a good correlation between inhibitory activity towards the mammalian 5-lipoxygenase and soyabean lipoxygenase (15-lipoxygenase) [4]. Inflammation is significantly involved in neurogenerative disorders leading to cognitive impairment, hence this assay was performed on BacoMind® to partly elucidate its anti-inflammatory potential. Please refer FIG. 3

### REFERENCES

1. Gaffney B J. (1996), Annu Rev Biophys Biomol Struct 25, 431-459.
2. Yamamoto S. (1992), Biochim Biophys Acta. 1128, 117-131.
3. Samuelsson B, Dahlén S, Lindgren J A, et al. (1987), Science. 237, 1171-1176.
4. Gleason M M, Rojas C J, Learn K S, Perrone M H, Bilder G E. (1995), American J. Physiology. 268 C: 1301-1307

### 2. DPPH Free Radical Scavenging Assay

Free radicals are generally very reactive molecules possessing an unpaired electron which are produced continuously in cells either as by-products of metabolism or by leakage from mitochondrial respiration (De Zwartt et. al 1999) [1]. The free radicals produced in-vivo include the active oxygen species such as super-oxide radical O₂⁻, hydrogen peroxide (H₂O₂) and hypochlorous acid (HOCl).

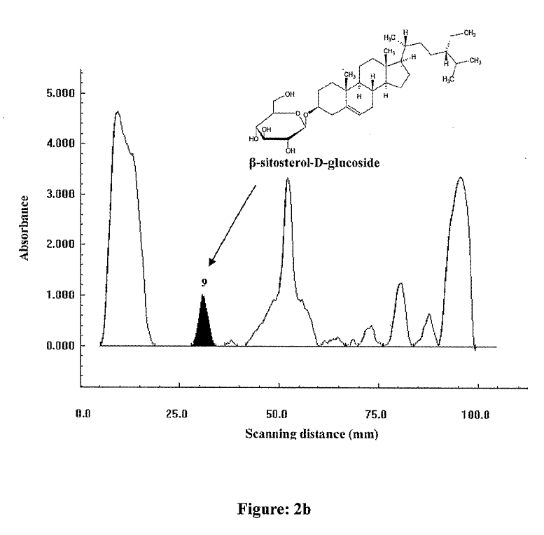

Oxygen free radicals have been shown to be responsible for many pathological conditions [2]. Free radicals and Reactive Oxygen Species (ROS) cause DNA damage, lipid per-oxidation, protein damage. They are known to be involved in the pathogenesis of a wide variety of clinical disorders such as cancer, cardiovascular diseases, inflammatory diseases, asthma and aging (Slater 1984; Vani et. al 1997) [3,4]. Free radicals like the hydroxyl radical, hydrogen peroxide, superoxide anion mediate components of the inflammatory response, with production of migratory factors, cyclic nucleotides and eicosanoids. Superoxide radicals amplify the inflammation process, increasing vascular permeability, adhesion of polymorphonuclear leucocytes to the endothelium and stimulation of platelet aggregation [2]. Free radicals are also involved in the pathogenesis of neurodegenerative disorders, hence this assay was performed to partly elucidate the anti-radical potential of Bacomind®. Please refer FIG. 4.

### REFERENCES

1. De Zwart L L, Meerman J H N; Commandeur J N M, Vermeulen N P E (1999). Free Rad. Biol. Med 26(1/2), 202-226.
2. Hemant R. Jadhav and K. K Bhutani. (2002). Phytother. Res., 16:771-773.

### 3. ABTS Radical Scavenging Assay

Free radicals are generally very reactive molecules possessing an unpaired electron which are produced continuously in cells either as by-products of metabolism or by leakage from mitochondrial respiration [1]. The free radicals produced in-vivo include the active oxygen species such as super-oxide radical O₂⁻, hydrogen peroxide (H₂O₂) and hypochlorous acid (HOCl).

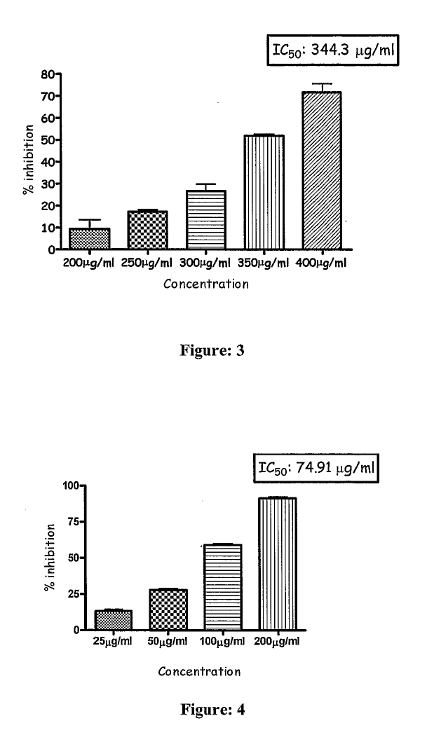

Oxygen free radicals have been shown to be responsible for many pathological conditions [2]. Free radicals and Reactive Oxygen Species (ROS) cause DNA damage, lipid per-oxidation, protein damage. They are known to be involved in the pathogenesis of a wide variety of clinical disorders as cancer, cardiovascular diseases, inflammatory diseases, asthma and aging [3, 4]. Free radicals like the hydroxyl radical, hydrogen peroxide, superoxide anion etc. mediate components of the inflammatory response, with production of migratory factors, cyclic nucleotides and eicosanoids. Superoxide radicals amplify the inflammation process, increasing vascular permeability, adhesion of polymorphonuclear leucocytes to the endothelium and stimulation of platelet aggregation [5]. Free radicals are also involved in the pathogenesis of neurodegenerative disorders, hence this assay was performed to partly elucidate the anti-radical potential of BacoMind®. Please refer FIG. 5.

### REFERENCES

1. De Zwart L L, Meerman J H, Commandeur J N, Vermeulen N P (1999). Free Rad. Biol. Med. 26(1-2): 202-226.
2. Jadhav H R, Bhutani K K (2002). Phytother. Res. 16:771-773.
3. Slater T F (1984). Biochem. J. 222:1-15.
4. Vani T, Rajini M, Sarkar S and Shishoo C J (1997) Int. J. Pharmac. 35(5): 313-317.
5. Aragon S M, Basabe B, Benedi J M, Villar A M (1998) Phytother. Res. 12:S104-S106

### 4. Butyrylcholinesterase Inhibition Assay

Acetylcholinesterase plays an important role in the central and peripheral nervous systems, along with the acetylcholine receptor, in the transmission of action potential across nerve-nerve and neuromuscular synapses. The enzyme's physiological task is the hydrolytic destruction of the cationic neurotransmitter, acetylcholine. Because of the pivotal role that acetylcholinesterase (AChE) plays in the nervous system, it has long been an attractive target for the rational design and discovery of mechanism-base inhibitors. Some inhibitors of acetylcholinesterase are known to be useful for the treatment of Alzheimer's disease, senile dementia, ataxia and for improving the long-term memory processes by enhancing cholinergic activity [1].

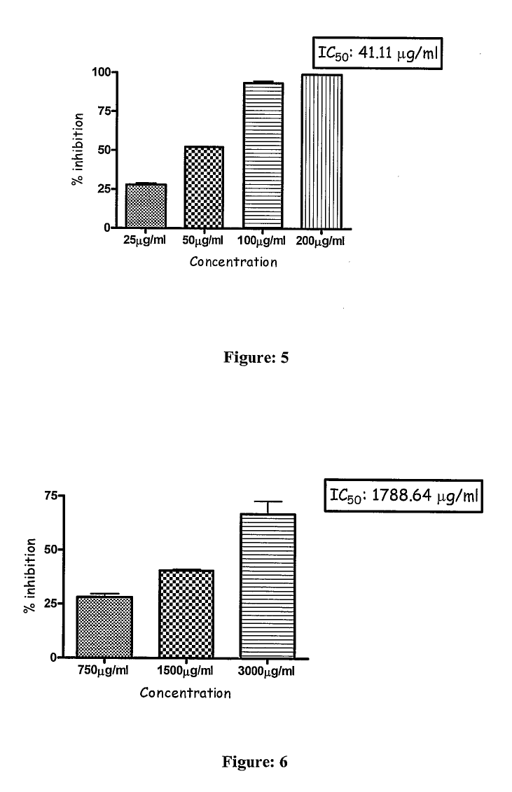

Butyrylcholinesterase (BuChE) is an enzyme that is related to AChE. It is expressed in glia, endothelial cells, and in neurons in selected areas of the central and peripheral nervous systems. Although the function of BuChE remain to be clearly defined, this enzyme is capable of catalyzing the hydrolysis of acetylcholine, and inhibition of BuChE leads to increased levels of this neurotransmitter in the brain [2]. Please refer FIG. 6

### REFERENCES

1. Atta-ur-Rahman (2001), Netherlands. Pp: 142-145.
2. Darvesh S, Walsh R, Kumar R, Caines A, Roberts S, Mager D, Rockwood K and Martin E (2003) Alzheimer's disease and associated disorders 17(2): 117-126.

### Example: 10

### Safety

The composition of BacoMind® given orally to Sprague-Dawley rats showed an LD₅₀ of 2400 mg/kg Reference may be made to In House Report No. 03, 1468. (2003), Acute oral toxicity of Bacopa monnieri extract to rat. Intox, Pune.

Both in-vitro and in-vivo studies clearly indicate the usefulness of the composition of BacoMind® in nootropic activity, neurodegenerative disorders, ADD/ADHD and learning and memory in slow learning children in particular. This prompted the applicant to initiate clinical trials on BacoMind® in memory impaired aged persons and improving memory function of the slow learners in children.

### Clinical Study 1

Aim: Efficacy and tolerability of the composition of BacoMind® on memory improvement in older persons—A double blind placebo controlled study
Objectives: The primary objective of the study was to evaluate the efficacy of the composition of BacoMind® as a health supplement, in reducing the symptoms of memory impairment in elderly individuals aged 50-75 years.

Secondary objectives of the study were:
To evaluate the safety and tolerability of the composition of BacoMind® in elderly individuals aged between 50-75 years, suffering from age associated memory impairment.
To do an overall assessment about the effectiveness of study medication to enhance memory and cognitive performance at a given dose following 12 weeks of supplementation in elderly people.
To study the effects following withdrawal of therapy i.e. from 12 to 24 weeks of duration.
To provide scientific evidence for the clinical use of the composition of BacoMind® supplementation as a cognition enhancer.

The study was conducted for a total duration of 24 weeks, where in the test substance/placebo administration period extended up to first 12 weeks; thereafter, no medication was administered for next 12 weeks following withdrawal of treatment. The study plan included total of 8 health visits. In the first visit, detailed medical examination including neuropsychological testing along with routine laboratory investigations was conducted. The synergistic composition of the present invention is a very effective butyrylcholinesterase inhibition.

### Neuropsychological Tests

The battery of neuropsychological tests included mini mental state examination (MMSE) and series of well established memory tests.

MMSE was developed by Indo-US-Cross-National Dementia Epidemiology Study. MMSE was evaluated at the baseline to assess the basic cognitive functions and to detect the intellectual deficits in participants and was scored in the range of 0-30 scale. The inclusion criteria also included the minimum score of MMSE as 24 and above for cognitive fitness as lesser scores were indicative of probable cognitive impairment such as dementia, Alzheimer's disease, etc.

A combination of well established auditory and visual neuropsychological tests was chosen to evaluate the speed of process information, attention and memory. The memory tests were designed by department of Psychiatry, Postgraduate Institute of Medical Education & Research (PGIMER), Chandigarh, India, modified for geriatric population. The construction and standardization of these tests were based on Rey Auditory Verbal Learning Test (AVLT) and Weschler Memory Scale (WMS III). All the tests were explained and performed by the psychologist in local language to the participants. The memory tests were repeated at 12 and 24 weeks again.

### Results

Out of 65 elderly volunteers (42 males & 23 females) enrolled; three candidates (1 from placebo and 2 from BacoMind®) didn't come for the second visit so they were not issued any medication. Finally 59 volunteers completed the study.

### Neuropsychological Tests

The results indicated that following 12 weeks of BacoMind® supplementation, improvements were seen in certain domains of memory.

### Attention:

A tendency of cognitive improvement in cancellation time was observed compared to other tests related to attention. There was a significant reduction in time required for cancellation test from baseline value of 190.70±9.09 sec to 171.20±10.56 sec (p<0.05) and 166.30±9.77 sec (p<0.01) at 12 and 24 weeks respectively. The improvement was 10.23% at 12 weeks with BacoMind™ treatment which extended to additional 2.56% over next 12 weeks following discontinuation of treatment.

### Memory:

Memory improvement was remarkable in different tests performed following BacoMind® supplementation. Delayed recall (DR) component of list learning test showed 26.97%) improvement at 12 weeks which further increased to 78.95% (p<0.01) at 24 weeks compared to baseline values and was found to be higher than placebo group (6.95% and 49.73%) improvement at 12th and 24th weeks respectively). Similarly the Delayed recall (DR) component of paired associates dissimilar test at 24 weeks showed significant improvement (p<0.01) in BacoMind® group (61.58%) compared to that of placebo group (43.50%).

BacoMind® also helped in better visual retention of memory as observed in visual retention test-1, at 12 weeks (14.29%) compared to placebo (8.08%). The cognitive improvement was significant in the performance results of 24^{th} week than that of 12^{th} week compared to that of 0^{th} week in both treatment groups. The compliance of study medication was satisfactory.

In summary the study findings suggest that Bacopa monnieri—Composition of BacoMind® may exert positive effects on cognitive process such as attention and long term memory component.

In summary, the study findings suggested that BacoMind®, revealed positive effects on cognitive process such as attention, short term and long term memory component. BacoMind® helped in improving the long term component of verbal memory as well as visual memory in elderly individuals indicating similarity to earlier published clinical studies on B. monnieri.

### Example: 11

### Clinical Study 2

Aim: Study of the composition of BacoMind® in Slow Learners or dull normal children

Efficacy of BacoMind® on memory improvement in children requiring individual education programme.

### Introduction:

The children requiring Individual Education Programme (IEP) are a group of children having Intelligence Quotient (IQ) ranging between 70-90. There are various factors (environmental and/or genetic) causing brain insult, resulting in brain dysfunction. The children requiring IEP could also have attention deficit disorders associated with hyperactivity, restlessness and lack of concentration. Such behavioral problems may be the contributing factors in case of some children requiring IEP. The present study was conducted to check the efficacy of BacoMind™, in improving memory function of the children requiring IEP.

### Study Design in Brief:

The clinical trial was open labeled, conducted in about 24 children, who on psychological evaluation revealed the I.Q. between 70-90 of the composition of BacoMind® was formulated as capsules of 225 mg strength and each child was administered with a single capsule every day.

The specific Memory Scale Test comprising of 10 sub-tests was conducted along with the standard battery of psychological tests. The pre & post-treatment evaluation was conducted by the same psychologist who also recorded the periodic (monthly) feedback from the parents about the scholastic performance of these children in the school, as well as the change in cognitive and behavioral function. BacoMind® was given for a period of 4-6 months and the memory test scores were completed in 24 children.

As shown in Table 3, the age range of children varied from 4 to 18 years, and the emphasis was given to younger age group considering brain-developing potentiality during earlier period.

The three age groups were made for the ease of psychological assessment and there were 12 Males and 12 Females. The children were evaluated to check the efficacy of the composition of BacoMind®.

TABLE 3
Clinical Profile of slow learner children under study

| Age Group (N = 24) | Males = 12 | Females = 12 |
|---|---|---|
| Below 10 years (9) | 05 | 04 |
| 10-14 years (13) | 06 | 07 |
| Above 14 years (02) | 01 | 01 |

### Statistics:

The values were expressed either as percentage or mean±SEM. The scores of performance of memory tests conducted in pre & post (0 & 6 months) treatment period were analyzed by student's paired‘t’ test application. The statistical significance was set at p≦0.05.

### Results

The analysis of data at the end of 6 months of treatment was conducted and discussed below in terms of total percentage improvement in the test scores and the number of children showing improvement in various areas of memory function. The total score of 10 memory subtests was studied in terms of percentage increase in scores, based on which 4 categories were made as shown below (Table no. 4).

TABLE 4
Improvement in Total Score of Memory Scale Test (N = 24)

| | I = ±/ | | | |
|---|---|---|---|---|
| Groups | No change | II = 20% | III = 21-50% | IV = 50-75% |
| No. of children | 4 | 13 | 5 | 2 |
| (N = 24) | | | | |
| Percentage of | 17% | 54% | 21% | 8% |
| children | | | | |

As the Memory Scale for children was designed to assess different aspects of memory, the test comprised of 10 subtests. The various areas of memory tests were highlighted in Table 5. The details of tests, method of administration and scoring system were already given at the initiation of the project, when the study design was submitted.

TABLE 5
Analysis Subtests of Memory Scale of Children on BacoMind ®

| | No. of | | |
|---|---|---|---|
| | children | % of | |
| Name of Subtest | N = 24 | children | Remark |
| ST1- Information | 6 | 25 | * |
| ST2- Orientation | 4 | 17 | * |
| ST3- Mental Control | 11 | 46 | ** |
| ST4- Digit Span | 17 | 71 | *** |
| ST5- Repeating words | 17 | 71 | *** |
| ST6- Repeating Sentences | 13 | 54 | *** |
| ST7- Verbal retention of Similar word pairs | 7 | 29 | ** |
| ST8- Verbal retention of Dissimilar word | 6 | 25 | * |
| pairs | | | |
| ST9- Visual Reproduction | 14 | 58 | *** |
| ST10- Recognition | 7 | 29 | ** |

% of children showing improvement on Memory Scale Test:
* 0-25%;
** 26-50%;
*** 51-75%.

The performance of slow learners in various memory tests were highlighted in Table 6.

TABLE 6
Improvement in memory test scores in
BacoMind ™ treated children requiring IEP

| | | Test Score | Test Score | |
|---|---|---|---|---|
| | | before | after | |
| Sl. | | treatment | treatment | Percentage |
| No. | Test | (0 month) | (6 month) | improvement |
| ST1 | Information | 4.58 ± 0.13 | 4.83 ± 0.08^{b} | 5.46 |
| ST2 | Orientation | 4.50 ± 0.18 | 4.71 ± 0.13 | 4.67 |
| ST3 | Mental Control | 3.71 ± 0.33 | 4.46 ± 0.30^{c} | 20.22 |
| ST4 | Digit Span | 5.21 ± 0.32 | 6.38 ± 0.25^{c} | 22.46 |
| ST5 | Repeating Words | 5.33 ± 0.44 | 6.54 ± 0.35^{c} | 22.70 |
| ST6 | Repeating Sentences | 3.21 ± 0.31 | 4.04 ± 0.23^{c} | 25.86 |
| ST7 | Verbal retention of | 4.25 ± 0.21 | 4.63 ± 0.16^{a} | 8.94 |
| | similar word pairs | | | |
| ST8 | Verbal retention of | 2.88 ± 0.32 | 3.13 ± 0.30^{b} | 8.68 |
| | dissimilar word pairs | | | |
| ST9 | Visual Reproduction | 5.00 ± 0.43 | 5.83 ± 0.36^{c} | 16.60 |
| ST10 | Recognition | 8.21 ± 0.36 | 8.33 ± 0.36 | 1.46 |

Mean ± SEM; n = 24,
ST—Sub Test
^{a}p ≦ 0.05;
^{b}p ≦ 0.01;
^{c}p ≦ 0.001 significant Vs before treatment value.

### Discussion

The percentage improvement in the total score of memory tests was divided into 4 groups as shown in Table 4. It was observed that 54.17% of children have shown up to 20% increase in improvement in the total score, while 20.83% showed between 21-50% increase. Interestingly, 8.33% of the children requiring IEP revealed remarkable (51-75%) increase in the total score.

The composition of BacoMind® had proved that a maximum (50%) children showing 20%) increase in improvement, and followed by 25% children showing improvement up to 50% increase in total score.

The immediate memory in terms of Digit Span (ST4), Short term memory for verbal recall of words (ST5), logical memory (ST6) and non-verbal (graphic) material for visual reproduction (ST9) were the 4 main areas showing the significant (more than 50%) of children) improvement in subtest scores. It was followed by the second group (25-50%) of children showing improvement in ST1, ST3, ST7, ST8 and ST10, which mainly involved the memory functions related to information, logical memory (verbal recognition and recall), visual and auditory memory tasks. The shorter time factor for recognition and recall was involved in subtest ST10, indicating the immediate memory function (Table 5 and 6). The least improvement was observed (16.67% of children) in memory tasks related to orientation (ST2) which checks the child's orientation to person, place and time forming the important part of memory.

Totally four children showed no improvement. In this 2 children remained static on the total score and 2 children showed reduction on the total score which could be explained as non cooperativeness of the children during the assessment session or other factors if any reported by the psychologists. Overall, the results revealed that there was significant improvement in total score of memory tests.

### Conclusion

The present study was based on neuropsychological evaluation of memory involving different aspects of memory function such as, immediate recall, audio and visual memory, recognition and retrieval, verbal and motor perceptual development in the children requiring IEP, and pre and post test substance comparison of the trial/study results. BacoMind®, treatment revealed a significant improvement in various memory related tasks in the children requiring IEP, regardless of the underlying factors causing brain damage (genetic or non genetic). The results supported the earlier findings of different studies on B. monnieri, claiming its efficacy in enhancing intellect and memory functions. The novel herbal supplement, BacoMind®, showed good results with no major side effects.

It is essential to study in depth, the nootropic activity of BacoMind®, at the cellular and molecular level using the non-invasive brain functional imaging techniques in human, so that the precise mechanism of action can be unraveled and the benefit of the medicinal plant can be available to the wide population.

### Dose

As explained earlier the composition can be in the form of capsules, tablets, syrups, liquids, lozenges etc for the purpose of administration. The dose of the present composition may vary accordingly to the requirement of the patients. The dose may preferably 100 mg to 3000 mg per day per adult as single or divided doses according to the condition

### ADVANTAGES OF PRESENT INVENTION

The composition is palatable & safe
The composition is effective in small dose and can also be administrated in any dosage form like capsules, tablets, syrups, lozenges etc.
The composition is useful in learning, memory and cognition
The composition can be mixed with the regular food substances like pizza, bread, health drinks, biscuits, chocolates
The composition may be administered to humans between 0.1 g to 3 g per day in divided dose or in single dose.

Based on the above in-vitro, in-vivo, safety and clinical studies it is evident that the present invention/composition is a synergistic herbal composition and useful in cognition, learning and memory.

I claim:

1. A synergistic herbal composition obtained from plant Bacopa species for management of neurodegenerative disorders, said composition comprising bacoside A3 at a concentration ranging from 0.1 to 25% w/w, bacopaside II at a concentration ranging from 0.1 to 25% w/w, jujubogenin isomer of bacopasaponin C at a concentration ranging from 0.1 to 25% w/w, bacopasaponin C at a concentration ranging from 0.1 to 25% w/w, bacopaside I at a concentration ranging from 0.1 to 25% w/w, bacosine at a concentration ranging from 0.1 to 25% w/w, apigenin at a concentration ranging from 0.05 to 5% w/w, sitosterol-D-glucoside at a concentration ranging from 0.05 to 5% w/w, and luteolin at a concentration ranging from 0.05 to 5% w/w of the composition optionally along with pharmaceutically acceptable excipients.

2. The synergistic herbal composition as claimed in claim 1, wherein the concentration of bacoside A3 is preferably about 5.7% w/w, bacopaside II is preferably about 5.6% w/w, jujubogenin isomer of bacopasaponin C is preferably about 8.2% w/w, bacopasaponin C is preferably about 5.4% w/w, bacopaside I is preferably about 7.1% w/w, bacosine is preferably about 1.9%, apigenin is preferably about 0.3%, sitosterol-D-glucoside is preferably about 0.9% and luteolin is preferably about 0.5%.

3. The synergistic herbal composition as claimed in claim 1, wherein the Bacopa species is Bacopa monnieri.

4. The synergistic herbal composition as claimed in claim 1, wherein said neurodegenerative disorders comprise attention deficit disorders, attention deficit hyperactivity disorders, dementia, amnesia, Alzheimer's disease cognition and slow learning.

5. The synergistic herbal composition as claimed in claim 1, wherein said pharmaceutically acceptable excipients are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

6. The synergistic herbal composition as claimed in claim 1, wherein the composition is formulated into dosage forms selected from a group comprising tablet troches, lozenges, aqueous or oily suspensions, dispersible powders or granules, emulsion in hard or soft gel capsules, syrups, elixirs.

7. The synergistic herbal composition as claimed in claim 1, wherein said composition is non-toxic and free of side effects.

8. A process for preparation of synergistic herbal composition from plant Bacopu monnieri for management of neurodegenerative disorders, said process comprising steps of:
(i) powdering the plant parts to obtain powder;
(ii) extracting the powder using solvent to obtain an extract;
(iii) refluxing followed by concentrating the extract to obtain a residue;
(iv) centrifuging the residue in a solvent to separate soluble and insoluble materials; and
(v) drying the insoluble material to obtain synergistic herbal composition comprising Bacoside A3 at a concentration ranging from 0.1 to 25% w/w, Bacopaside II at a concentration ranging from 0.1 to 25% w/w, Jujubogenin isomer of bacopasaponin C at a concentration ranging from 0.1 to 25% w/w, Bacopasaponin C at a concentration ranging from 0.1 to 25% w/w, Bacopaside I at a concentration ranging from 0.1 to 25% w/w, Bacosine at a concentration ranging from 0.1 to 25% w/w, Apigenin at a concentration ranging from 0.05 to 5% w/w, Sitosterol-D-glucoside at a concentration ranging from 0.05 to 5% w/w, and Luteolin at a concentration ranging from 0.05 to 5% w/w of the composition.

9. The process as claimed in claim 8, wherein said plant parts are selected from a group comprising root, shoot, seeds and leaves or the whole plant.

10. The process as claimed in claim 8, wherein the plant pans are powdered to obtain particle size ranging from 30 mesh to 50 mesh and preferably of 40 mesh.

11. The process as claimed in claim 8, wherein said powder is extracted using alcoholic solvent selected from a group comprising ethanol methanol, propanol and isopropanol.

12. The process as claimed in claim 8, wherein said powder is extracted preferably using methanol.

13. The process as claimed in claim 8, wherein said extract is refluxed at temperature ranging from 60 to 80° C. and for time period ranging from 1 to 6 hours.

14. The process as claimed in claim 8, wherein said extract is refluxed at temperature preferably about 70° C. and preferably for time period of about 4 hours.

15. The process as claimed in claim 8, wherein said extract is concentrated under vacuum to obtain concentrated residue.

16. The process as claimed in claim 8, wherein the insoluble material is dried at a temperature less than 75° C.

17. Use of a synergistic herbal composition comprising Bacoside A3 at a concentration ranging from 0.1 to 25% w/w, Bacopaside II at a concentration ranging from 0.1 to 25% w/w, Jujubogenin isomer of bacopasaponin C at a concentration ranging from 0.1 to 25% w/w, Bacopasaponin C at a concentration ranging from 0.1 to 25% w/w, Bacopaside I at a concentration ranging from 0.1 to 25% w/w, Bacosine at a concentration ranging from 0.1 so 25% w/w, Apigenin at a concentration ranging from 0.05 to 5% w/w, Sitosterol-D-glucoside at a concentration ranging from 0.05 to 5% w/w, and Luteolin at a concentration ranging from 0.05 to 5% w/w of the composition to manufacture a medicament for management of neurodegenerative disorders in a subject in need thereof.

18. The use as claimed in claim 17, wherein the subject is animal including human being.

19. The use as claimed in claim 17, wherein the composition is administered at a dose ranging from 100 mg to 3000 mg per day or in divided doses or in single dose in a subject in need thereof.

20. The use as claimed in claim 17, wherein the composition is free of adverse effects.
